(19) United States
(12) Patent Application Publication
(10) Pub. No.: US 2016/0354602 A1
(43) Pub. Date: Dec. 8, 2016

(54) UPPER AIRWAY STIMULATOR SYSTEMS FOR OBSTRUCTIVE SLEEP APNEA

(71) Applicant: THE ALFRED E. MANN FOUNDATION FOR SCIENTIFIC RESEARCH, Santa Clara, CA (US)

(72) Inventors: DESMOND B. KEENAN, VALENCIA, CA (US); MORTEN HANSEN, VALENCIA, CA (US); BRIAN R. DEARDEN, PASADENA, CA (US); SIEGMAR SCHMIDT, SIMI VALLEY, CA (US); WILLIAM A. DAI, PORTER RANCH, CA (US)

(73) Assignee: THE ALFRED E. MANN FOUNDATION FOR SCIENTIFIC RESEARCH, SANTA CLARITA, CA (US)

(21) Appl. No.: 15/093,600

(22) Filed: Apr. 7, 2016

Related U.S. Application Data
(60) Provisional application No. 62/171,531, filed on Jun. 5, 2015.
(60) Provisional application No. 62/171,608, filed on Jun. 5, 2015.

(51) Int. Cl. A61N 1/36

(52) U.S. Cl. CPC A61N 1/3601; A61N 1/3611; A61N 1/36139

(57) ABSTRACT

An upper airway stimulator for treating obstructive sleep apnea is described. In some embodiments, the upper airway stimulator monitors the phase difference between ribcage expansion and abdomen expansion to detect apneic events and stimulates to alleviate those events. In some embodiments, the upper airway stimulator applies primary stimulation when an apneic event is not detected and secondary stimulation when an apneic event is detected. In some embodiments, the upper airway stimulator applies primary stimulation when the patient is not in an apneic position and secondary stimulation when the patient is in an apneic position.

### CROSS REFERENCE TO RELATED APPLICATIONS

The present application claims benefit under 35 U.S.C. §119 (e) to U.S. provisional application Ser. No.:62/171,531 filed on Jun. 5, 2015 and to U.S. provisional application Ser. No.: 62/171,608 filed on Jun. 5, 2015, which are incorporated herein by reference in their entirety.

### BACKGROUND

Obstructive sleep apnea is a disease in which the upper airway of a patient can become obstructed (apnea) or partially obstructed (hypopnea) during sleep. It is highly prevalent and has serious effects and comorbidities.

The use of neurostimulators to open the upper airway, thereby alleviating apneic events, is being explored. Currently available systems do not provide sufficient relief from the disease. Accordingly, there remains a need for improved techniques and systems for treating obstructive sleep apnea.

### SUMMARY

One aspect of the present disclosure relates to a system for treating obstructive sleep apnea. The system comprises a first sensor configured to generate a first signal corresponding to movement of the ribcage of the patient during respiration and a second sensor configured to generate a second signal corresponding to movement of the abdomen of the patient during respiration. The system also comprises a stimulator configured to deliver stimulation to a nerve which innervates an upper airway muscle, such as the hypoglossal nerve. The system further comprises a controller coupled to the first sensor, the second sensor, and the stimulator. The controller is configured to receive the first signal from the first sensor and the second signal from the second sensor. The controller is further configured to cause the stimulator to stimulate the nerve based on whether the first signal and the second signal are out of phase.

Another aspect of the present disclosure relates to a method of treating obstructive sleep apnea. The method comprises acquiring a first signal corresponding to movement of the ribcage of the patient during respiration, acquiring a second signal corresponding to movement of the abdomen of the patient during respiration, determining whether the first signal and the second signal are out of phase, and stimulating a nerve innervating an upper airway muscle upon determining that the first signal and the second signal are out of phase.

Another aspect of the present disclosure relates to a system for treating obstructive sleep apnea. The system comprises an apnea sensor configured to generate an apnea signal. The system also comprises a stimulator configured to deliver stimulation to a nerve which innervates an upper airway muscle. The system further comprises a controller coupled to the apnea sensor and the stimulator. The controller is configured to determine whether an apneic event is detected based on the apnea signal. The controller is further configured to cause the stimulator to apply primary stimulation to the nerve if no apneic event is detected, and to cause the stimulator to apply secondary stimulation to the nerve upon detecting an apneic event.

Another aspect of the present disclosure relates to a method of treating obstructive sleep apnea. The method comprises acquiring an apnea signal, determining whether an apneic event is detected based on the apnea signal, applying primary stimulation to a nerve innervating an upper airway muscle when an apneic event is not detected, and applying secondary stimulation to the nerve innervating an upper airway muscle upon detecting an apneic event.

Another aspect of the present disclosure relates to a system for treating obstructive sleep apnea. The system comprises a body position sensor configured to generate a body position signal. The system also comprises a stimulator configured to deliver stimulation to a nerve which innervates an upper airway muscle. The system further comprises a controller coupled to the body position sensor and the stimulator. The controller is configured to receive the body position signal from the body position sensor and determine whether the patient is in an apneic position based on the body position signal. The controller is further configured to cause the stimulator to apply primary simulation to the nerve if the patient is not in an apneic position, and to cause the stimulator to apply secondary stimulation to the nerve upon determining that the patient is in an apneic position.

Another aspect of the present disclosure relates to a method of treating obstructive sleep apnea. The method comprises acquiring a body position signal, determining whether the patient is in an apneic position based on the body position signal, applying primary stimulation to a nerve innervating an upper airway muscle when the patient is not in an apneic position, and applying secondary stimulation to the nerve innervating an upper airway muscle upon determining that the patient is in an apneic position.

These and other features, aspects, and advantages of the present disclosure will become better understood with regard to the following description, appended claims, and accompanying drawings.

### BRIEF DESCRIPTION OF THE DRAWINGS

So that the present disclosure can be better understood, a detailed description is provided below that makes reference to features of various embodiments, some of which are illustrated in the accompanying drawings. The accompanying drawings, however, merely illustrate the more pertinent features of the present disclosure and are not intended to limit the scope of the claims. Some of the drawings may not depict all of the components of a given method or apparatus.

FIG. 1 shows an embodiment of a system for treating obstructive sleep apnea.

FIG. 2A is a side view of a patient showing the peaks of respiration in a patient undergoing unobstructed breathing.

FIG. 2B is a side view of a patient showing the peaks of respiration in a patient undergoing obstructed breathing.

FIG. 3 is a block diagram of an embodiment of a system for treating obstructive sleep apnea.

FIG. 4 is a block diagram of an embodiment of a system for treating obstructive sleep apnea which includes a separate sensing unit and stimulating unit.

FIG. 5 shows an embodiment of a system for treating obstructive sleep apnea implanted within the body of a patient.

FIG. 6 shows an embodiment of a system for treating obstructive sleep apnea implanted within the body of a patient.

FIG. 7 is a flowchart of a method of treating obstructive sleep apnea according to some embodiments.

FIG. 8 is a flowchart of a method of treating obstructive sleep apnea which includes primary and secondary forms of stimulation based on detected apneic events according to some embodiments.

FIG. 9 is a flowchart of a method of treating obstructive sleep apnea which includes primary and secondary forms of stimulation based on patient body position according to some embodiments.

### DETAILED DESCRIPTION

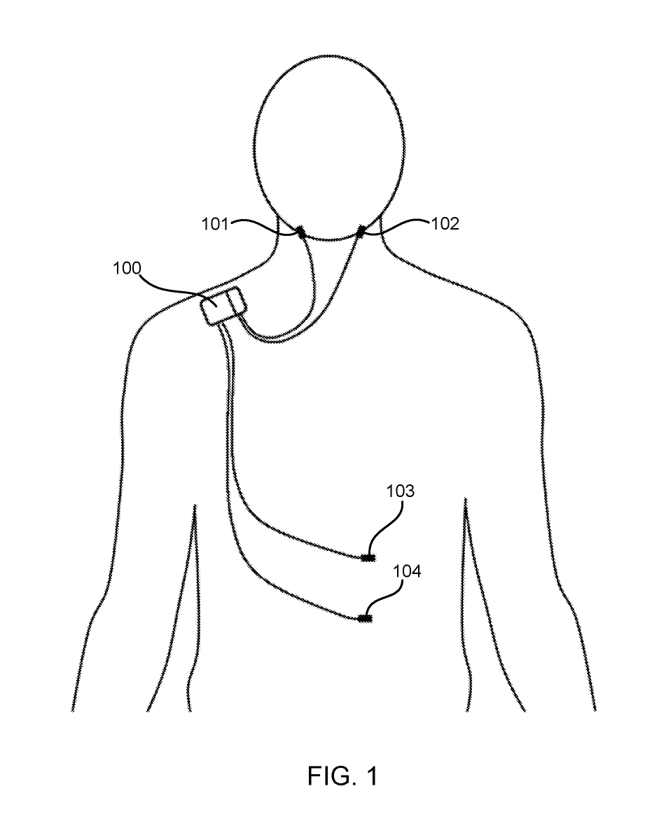

FIG. 1 is an embodiment of a system for treating obstructive sleep apnea. An implantable device 100 is implanted within the patient. The device includes a stimulator coupled to electrodes 101 and 102. Although two electrodes are shown, in some embodiments only one electrode is used. The electrodes 101 and 102 are positioned to stimulate nerves which innervate an upper airway muscle. In embodiments, the nerve is the hypoglossal nerve. In embodiments, the electrodes 101 and 102 are positioned to apply bilateral stimulation to the two branches of the hypoglossal nerve. In embodiments, the electrodes 101 and 102 are nerve cuff electrodes.

The implantable device 100 is also coupled to a first sensor 103 and a second sensor 104. The first sensor 103 is positioned at a point where it can detect movement or expansion of the ribcage. The second sensor 104 is positioned at a point where it can detect movement or expansion of the abdomen. The first sensor 103 and the second sensor 104 can be sensors positioned at sites remote from the implantable device 100, or may be electrodes coupled to sensors contained inside the implantable device 100. The first sensor 103 may be a bioimpedance sensor, an accelerometer, or a pressure sensor. The second sensor 104 may be a bioimpedance sensor, an accelerometer, or a pressure sensor. In an embodiment, the implantable device 100 comprises the first sensor 103 and is implanted at a position where the first sensor 103 can detect movement or expansion of the ribcage. In an embodiment, the first sensor 103 is an accelerometer or other non-contact motion sensor contained inside the implantable device 100.

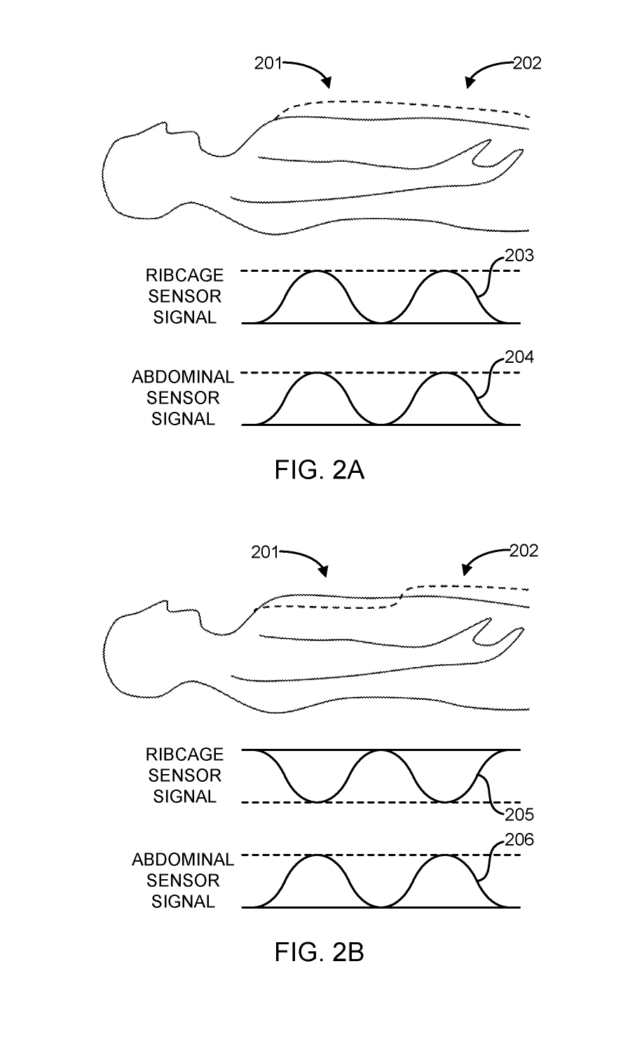

FIG. 2A shows the relationship between expansion of the ribcage 201 due to respiration and expansion of the abdomen 202 due to respiration in a person undergoing unobstructed breathing. The solid line represents the resting state. The dotted line represents the end of inspiration. During inspiration, both the ribcage 201 and the abdomen 202 expand outward up until the end of inspiration. During expiration, both the ribcage 201 and the abdomen 202 recede to the resting position. Accordingly, sensors positioned to detect movement or expansion of the ribcage 201 and abdomen 202, such as the sensors 103 and 104 of FIG. 1, will generate signals 203 and 204 which are in phase.

FIG. 2B shows the relationship between expansion of the ribcage 201 due to respiration and expansion of the abdomen 202 due to respiration in a person undergoing obstructed breathing. This relationship is present when the upper airway is blocked, as is the case when an apneic event is occurring. The solid line represents the resting state. The dotted line represents the end of inspiration. The abdomen 202 expands during inspiration and recedes to the resting state during expiration, in the same way as during unobstructed respiration. The ribcage 201, however, contracts during the inspiratory portion of the respiration cycle and expands back to the resting state during the expiratory portion of the respiration cycle. Accordingly, sensors positioned to detect movement or expansion of the ribcage 201 and abdomen 202, such as the sensors 103 and 104 of FIG. 1, will generate signals 205 and 206 which are 180 degrees out of phase. In some situations, the signals representative of expansion of the ribcage and expansion of the abdomen may be out of phase during an apnea or hypopnea, but less than 180 degrees out of phase. This may occur during less severe obstructions.

The implantable device 100 is configured to receive the signals from the first sensor 103 and the second sensor 104. By comparing the phase of the two signals, the implantable device 100 may detect when an apneic event is occurring in the patient. It may then deliver treatment based on this information.

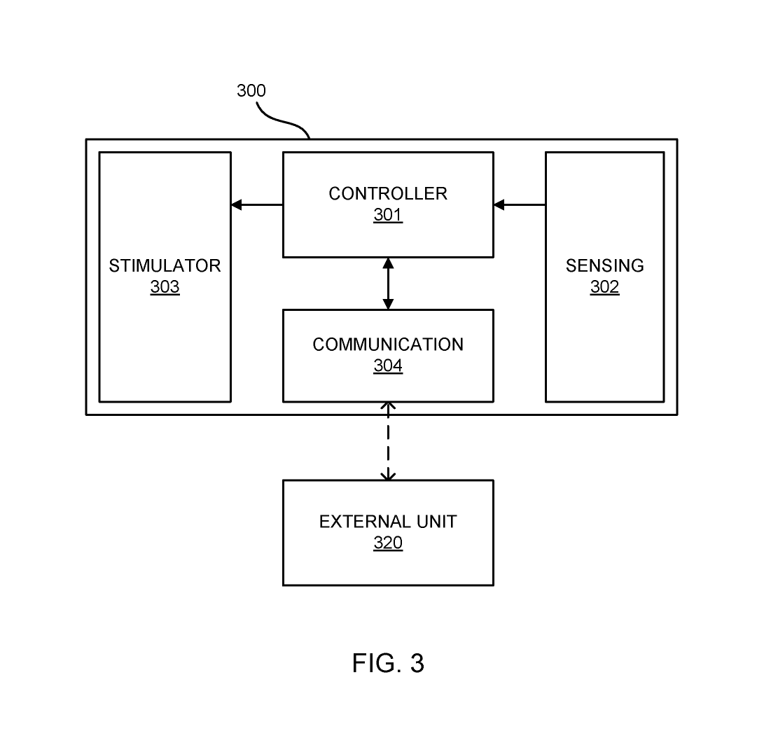

FIG. 3 is a block diagram of an embodiment of a system for treating obstructive sleep apnea. It includes an implantable unit 300 and an external unit 320. The implantable unit 300 includes a controller 301, a sensing system 302, a stimulator 303, and a communication system 304.

The sensing system 302 is configured to acquire signals related to respiration. In some embodiments, the sensing system 302 generates two separate signals—one representing the movement or expansion of the patient's ribcage due to respiration, and one representing movement or expansion of the patient's abdomen due to respiration. The sensing system 302 may acquire these signals using various sensors, including accelerometers, bioimpedance sensors, or pressure sensors, or some combination thereof. The signals are passed to the controller 301.

The stimulator 303 is configured to deliver stimulation to a nerve innervating the upper airway of the patient through electrodes implanted proximate the nerve. In embodiments, the nerve is the hypoglossal nerve. In embodiments, the upper airway muscle comprises the genioglossus or the geniohyoid or some combination thereof. When the nerve is stimulated, it activates the upper airway muscle, thereby preventing or alleviating obstructive apneic events. In some embodiments, the intensity of the stimulation applied to the nerve is sufficient to promote tonus in the upper airway muscle. In some embodiments, the intensity of the stimulation applied to the nerve causes bulk muscle movement in the upper airway muscle. The stimulator 303 is coupled to the controller 301. The controller 301 controls when the stimulator 303 applies stimulation. In some embodiments, the controller 301 can control the intensity of the stimulation applied by the stimulator 303. In some embodiments, the intensity of the stimulation applied by stimulator 303 may be varied by changing the amplitude, pulse width, or frequency of the stimulation.

In some embodiments, the controller 301 is configured to receive two respiration signals representing the movement or expansion of the ribcage and the abdomen of the patient from the sensing system 302 and monitor the phase difference between the two. The controller 301 causes the stimulator 303 to stimulate based on this phase difference. In embodiments, if the two signals are out of phase, signifying an apneic event, the controller 301 causes the stimulator 303 to apply stimulation to the nerve to alleviate the apneic event. Note that by “out of phase” it can be meant substantially out of phase. Biological signals are not perfect waveforms and include substantial noise. Accordingly, the two respiration signals are not likely to ever be perfectly in phase in the literal sense of the term. Phase difference due to biological imperfections and noise, however, will be distinguishable from phase differences present during obstructive apneic events, which can approach 180 degrees at full obstruction—in embodiments, “out of phase” refers to these phase differences. The controller 301 monitors the phase difference between the two signals in order to detect when the phase difference becomes substantial enough that the difference is likely due to an apneic event which is occurring or is about to occur. In some embodiments, the controller 301 controls the intensity of the stimulation based on the phase difference between the two signals. In an embodiment, the controller 301 controls the stimulator 303 to apply higher intensity stimulation for higher phase difference.

In embodiments, the controller 301 is configured to determine when the patient is in the inspiratory portion of the respiratory cycle—where the patient is breathing in or attempting to breathe in. The controller 301 may condition the application of stimulation upon the patient being in this inspiratory phase of respiration. The controller 301 causing the stimulator 303 to stimulate can, therefore, mean applying stimulation during these inspiratory portions of the respiration cycle (or applying stimulation starting slightly before the inspiration and ending at the end of inspiration), and not the remainder of the respiration cycle, when all other conditions for stimulation are met. This can be accomplished by monitoring the first and second signals, especially the second signal.

In embodiments, the sensing system 302 includes a body position sensor. The body position sensor may be an accelerometer, a gyroscope, or a combination of an accelerometer and a gyroscope. The body position sensor generates a signal related to the orientation of the patient's body and passes that signal to the controller 301. The controller 301 monitors this signal to determine the orientation of the patient's body. In embodiments using an accelerometer as a body position sensor, the controller 301 may monitor the signal from the accelerometer for the DC portion of the signal corresponding to gravity to determine the orientation of the patient's body. In embodiments using a gyroscope, the controller 301 may monitor the signal from the gyroscope to track rotation of the patient from one position to another. In embodiments, the controller activates the portions of the sensing system 302 which monitor ribcage and abdomen respiration when the orientation of the patient's body indicates that the patient is in an apneic position. An apneic position is a position in which the patient is likely to experience apneic events. The most common apneic position is supine, but can include left side, right side, or both. Patients with positional sleep apnea experience significantly more apneic events while in particular apneic positions. This can allow the device to preserve battery life by monitoring respiration only when the patient is likely to experience apneic events. In some embodiments, the controller 301 includes a memory. This memory is configured to be programmed to contain positional sleep apnea data for the patient. In embodiments, the memory is programmed pre-implantation, or post implantation using the external unit 320, with positional sleep apnea data for the patient, wherein said positional sleep apnea data may have been generated from a sleep study of the patient. When the controller 301 is determining whether the patient is in an apneic position, the controller 301 may consult the information stored on this memory in addition to the body position signal.

In embodiments, the sensing system 302 includes a sleep sensor. The sleep sensor may comprise sensors used in polysomnography, such as an EMG sensor across the jaw line, an EEG sensor, and an EOG sensor. The sleep sensor may additionally or alternatively comprise an accelerometer or other activity sensor, or a temperature sensor. The sleep sensor generates a sleep signal. The controller 301 monitors the sleep signal to determine when the patient is asleep and activates the sensing system 302 upon determining that the patient is asleep. In some embodiments, the sleep signal is a polysomnography signal and the controller evaluates the signal using techniques used in polysomnography. In some embodiments, especially embodiments utilizing an accelerometer, the sleep signal contains information about the orientation of the body of the patient and the controller 301 determines that the patient is asleep when the sleep signal indicates that the patient has been supine (or, alternatively, in any lying position) for a prolonged period. In some embodiments, especially embodiments utilizing an accelerometer or other activity sensor, the sleep signal contains information about the heart rate or breathing patterns of the patient and the controller 301 determines that the patient is asleep when the sleep signal indicates that the heart rate or breathing patterns of the patient are consistent with sleep. Respiration and heart rate typically exhibit less variability, both in amplitude and frequency, when a patient is in a sleep state. The controller 301 may, therefore, determine that the heart rate or breathing patterns of the patient are consistent with sleep by monitoring the sleep signal for a reduction in variance of the heart rate or breathing patterns. In embodiments utilizing a temperature sensor, the controller 301 determines that the patient is asleep when the sleep signal indicates that the temperature of the patient has decreased in a manner consistent with sleep. In some embodiments, the sleep sensor comprises a plurality of sensor types and the sleep signal comprises the data received from each of the plurality of sensor types. The controller 301 may conserve power, thereby extending battery life, by activating the ribcage and abdomen respiration monitoring portions of the sensing system 302 only when it determines that the patient is asleep. Additionally, by only monitoring ribcage and abdomen respiration when the controller 301 determines that the patient is asleep, the system can avoid the possibility of false-positive detection of an apneic event causing stimulation while the patient is awake. The controller 301 may take additional power conservation steps when it determines that the patient is not asleep. In an embodiment, the sleep sensor is external to the body of the patient, and the communication system 304 periodically wirelessly polls the sleep sensor to determine whether the patient is asleep. In embodiments, the controller 301 waits until the patient has been asleep for a set period of time before it will cause the stimulator 303 to stimulate.

In embodiments, the controller 301 monitors the variance in one or both of the ribcage and abdomen respiration signals received from the sensing system 302. The controller uses the variance to determine when the patient is asleep. Respiration typically exhibits less variability, both in amplitude and frequency, when a patient is in a sleep state. The controller 301 may, therefore, determine that the patient is asleep by monitoring for a reduction in the variance of one or more of breath-to-breath amplitude or breath-to-breath frequency of the first signal or the second signal. Low variance in the signals indicates that the patient is asleep, high variance indicates that the patient is awake. The controller 301 may not monitor the phase difference between the two signals or cause the stimulator 303 to stimulate the nerve unless the patient is asleep. The controller 301 may also wait until the patient has been asleep for a set period of time before it will cause the stimulator 303 to stimulate.

In some embodiments, the controller 301 is configured to monitor an apnea signal from the sensor system 302 to determine whether the patient is experiencing or about to experience an apneic event. The apnea signal may be the phase difference between the signal from the sensor monitoring expansion of the ribcage and the signal from the sensor monitoring expansion of the abdomen, as discussed above, though alternatives are contemplated and this embodiment should not be limited to that particular apnea signal. When the controller 301 does not detect that the patient is experiencing an apneic event, the controller 301 causes the stimulator 303 to apply primary stimulation (this stimulation may be applied during the inspiratory portion of respiration). Upon determining that the patient is experiencing or is about to experience an apneic event, the controller 301 causes the stimulator 303 to apply secondary stimulation (this stimulation may also be applied during the inspiratory portion of respiration). Several embodiments are contemplated for primary and secondary stimulation. These embodiments are discussed in detail below.

In embodiments, the sensing system 302 includes a body position sensor. The body position sensor may be an accelerometer or a gyroscope. The body position sensor generates a body position signal related to the orientation of the patient's body and passes that signal to the controller 301. The controller 301 is configured to monitor a body position signal from the body position sensor to determine whether the patient is in an apneic position. An apneic position is a position in which the patient is likely to experience apneic events. The most common apneic position is supine, but can include left side, right side, or both. Patients with positional sleep apnea experience significantly more apneic events while in particular apneic positions. When the controller 301 does not detect that the patient is in an apneic position, the controller 301 causes the stimulator 303 to apply primary stimulation (this stimulation may be applied during the inspiratory portion of respiration). Upon determining that the patient is in an apneic position, the controller 301 causes the stimulator 303 to apply secondary stimulation (this stimulation may also be applied during the inspiratory portion of respiration). Several embodiments are contemplated for primary and secondary stimulation. These embodiments are discussed in detail below. In some embodiments, the controller 301 includes a memory. This memory is configured to be programmed to contain positional sleep apnea data for the patient. When the controller 301 is determining whether the patient is in an apneic position, the controller 301 may consult the information stored on this memory in addition to the body position signal.

The communication system 304 is configured to communicate wirelessly with the external unit 320. The external unit 320 may be a clinician's programmer or a patient's remote. The external unit 320 may be used to configure the algorithms used by the controller to process the signals from the sensing system 302 and determine when to activate the stimulator 303. The external unit 320 transmits the necessary information to the communication system 304 and the communication system 304 passes it to the controller 301. This can include data regarding apneic positions in patients with positional sleep apnea, as discussed above. The communication system 304 may transmit status information to the external unit 320.

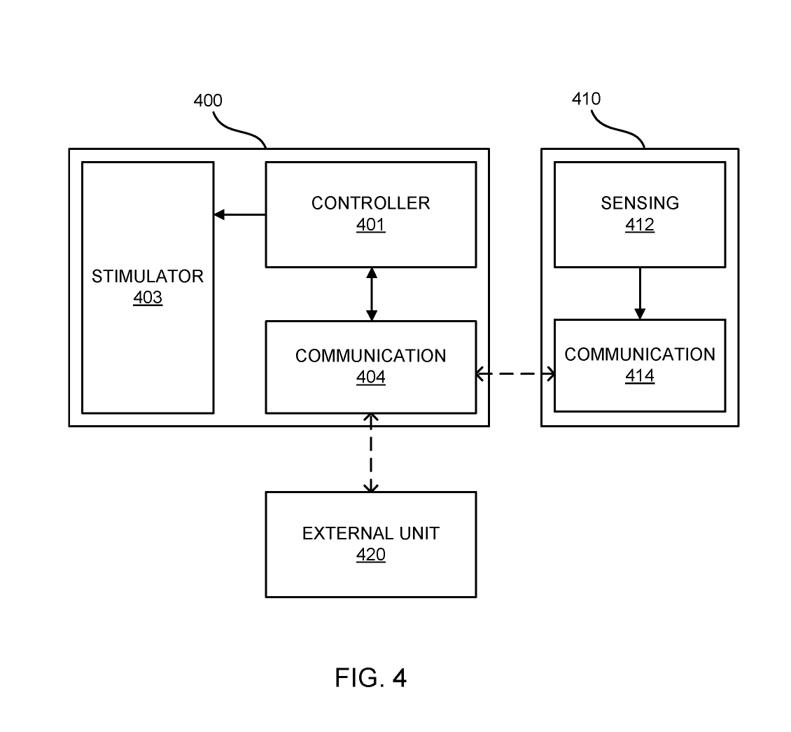

FIG. 4 is a block diagram of an alternative embodiment of a system for treating obstructive sleep apnea. It includes an implantable stimulator unit 400, a sensor unit 410, and an external unit 420.

The sensor unit 410 includes a sensing system 412 and a communication system 414. In preferred embodiments, the sensor unit 410 is also implantable. The sensing system 412 generates the first signal representative of movement of the ribcage due to respiration and the second signal representative of movement of the abdomen due to respiration. The sensing system may be generally configured as described in reference to the sensing system 302 of FIG. 3. The sensing system 412 passes the signals to sensor unit communication system 414. The sensor unit communication system 414 transmits the signals to the stimulator unit communication system 404.

The stimulator unit 400 includes a controller 401, a stimulator 403, and a communication system 404. The communication system 404 passes the signals representing ribcage and abdomen expansion to the controller 401. The controller 401 may use the communication system 404 to indicate to the sensor unit 410 when the signals should be measured. Otherwise, the controller 401, stimulator 403, and communication system 404 can generally be configured as described in reference to the controller 301, stimulator 303, and communication system 304 of FIG. 3, respectively.

In an alternative embodiment, the stimulator unit 400 also includes a sensing system. The stimulator unit sensing system is configured to generate the signal representative of expansion of the ribcage due to respiration, and the sensor unit sensing system is configured to generate the signal representative of expansion of the abdomen due to respiration.

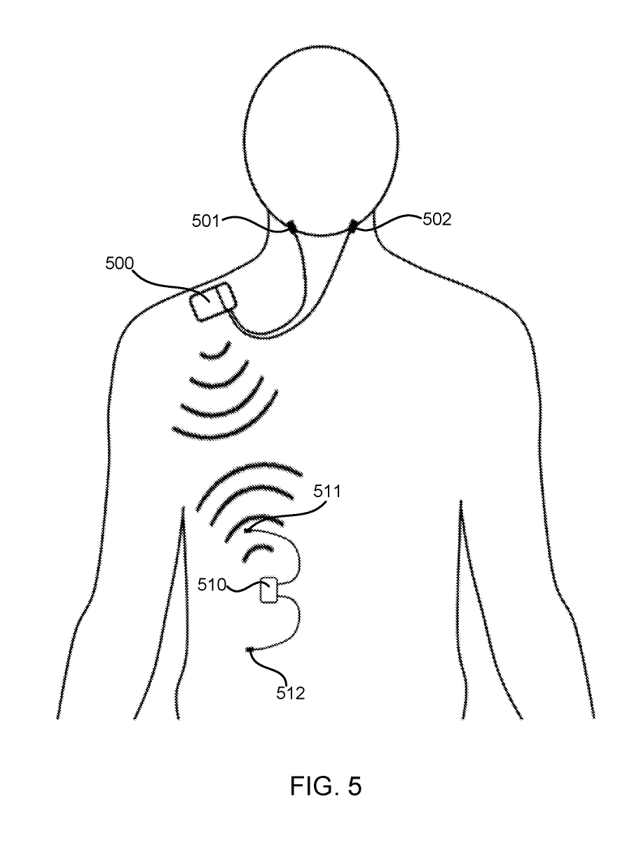

FIG. 5 is an embodiment of a system for treating obstructive sleep apnea comprising an implantable stimulator unit 500 and a sensor unit 510. The stimulator unit 500 is configured to apply stimulation to a nerve innervating an upper airway muscle through the electrodes 501 and 502. The sensor unit 510 is coupled to a first sensor 511 and a second sensor 512 and is configured to receive signals from those sensors. The first sensor 511 is configured to be placed in a position where it can detect expansion of the ribcage due to respiration. The second sensor 512 is configured to be placed in a position where it can detect expansion of the abdomen due to respiration. In embodiments, the sensor unit 510, the first sensor 511, and the second sensor 512 are implantable.

The sensor unit 510 and the stimulator unit 500 communicate wirelessly. This wireless communication can be directly between the implanted stimulator unit 500 and the sensor unit 510, can use an external unit as an intermediary, or can use an implanted transponder device as an intermediary between the two. In alternative embodiments, the sensor unit 510 has a wired connection with the stimulator unit 500 and the sensor unit 510 and stimulator unit 500 communicate through the wired connection.

The first sensor 511 may be a pressure sensor, an accelerometer, or a bioimpedance sensor. In embodiments in which the first sensor is a bioimpedance sensor, the impedance of body tissue between an electrode at 511 and an electrode located on the case of the sensor unit 510. The second sensor 512 may be a pressure sensor, an accelerometer, or a bioimpedance sensor. In embodiments in which the second sensor is a bioimpedance sensor, the impedance of body tissue between an electrode at 512 and an electrode located on the case of the sensor unit 510.

In an alternative embodiment, not pictured, the sensor unit 510 has only one lead, said lead having multiple electrodes, the sensor unit 510 includes an electrode located on its case, and the first sensor and the second sensor are bioimpedance sensors. The impedance of tissue between the sensor unit 510 and a proximal electrode is measured to acquire the first signal, and the impedance of tissue between the proximal electrode and a distal electrode is measured to acquire the second signal.

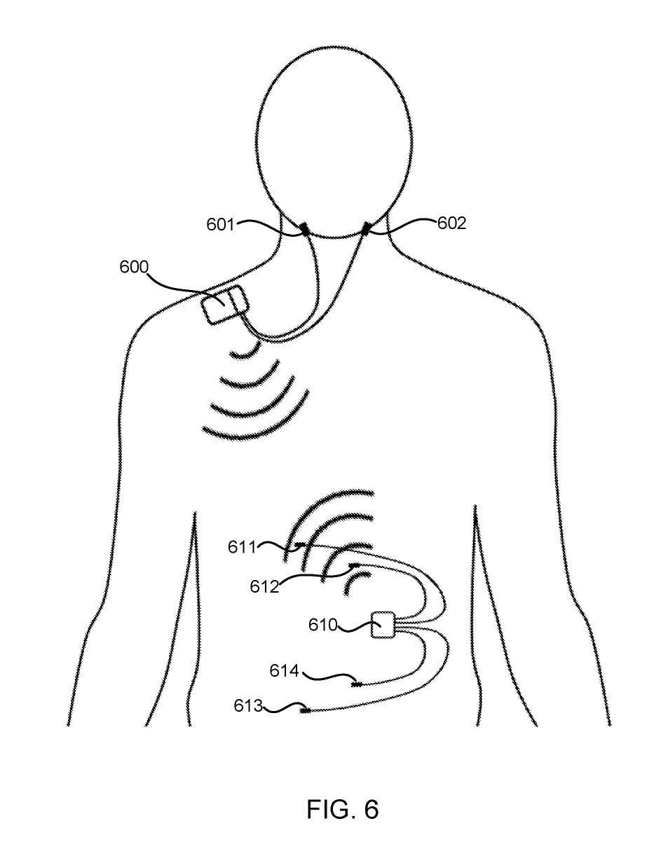

FIG. 6 is an embodiment of a system for treating obstructive sleep apnea comprising an implantable stimulator unit 500 and an implantable bioimpedance sensor unit 610. In this embodiment, the sensor unit 610 includes at least four electrodes. Electrode 611 and electrode 612 are positioned such that the tissue between the two electrodes is tissue which moves responsive to ribcage respiration. Preferably, electrode 611 is placed on the right side of the ribcage and electrode 612 is placed on the left side of the ribcage. The impedance of the tissue is measured to acquire a signal representative of expansion of the ribcage due to respiration. Electrode 613 and electrode 614 are positioned such that the tissue between the two electrodes is tissue which moves responsive to abdominal respiration. Preferably, electrode 613 is placed on the right side of the abdomen and electrode 614 is placed on the left side of the abdomen. The impedance of the tissue is measured to acquire a signal representative of expansion of the abdomen due to respiration.

The sensor unit 610 is shown as having four leads, one corresponding to each electrode. In an alternative embodiment, the sensor unit 610 has two leads, the first lead comprising electrodes 611 and 612, the second lead comprising electrodes 613 and 614.

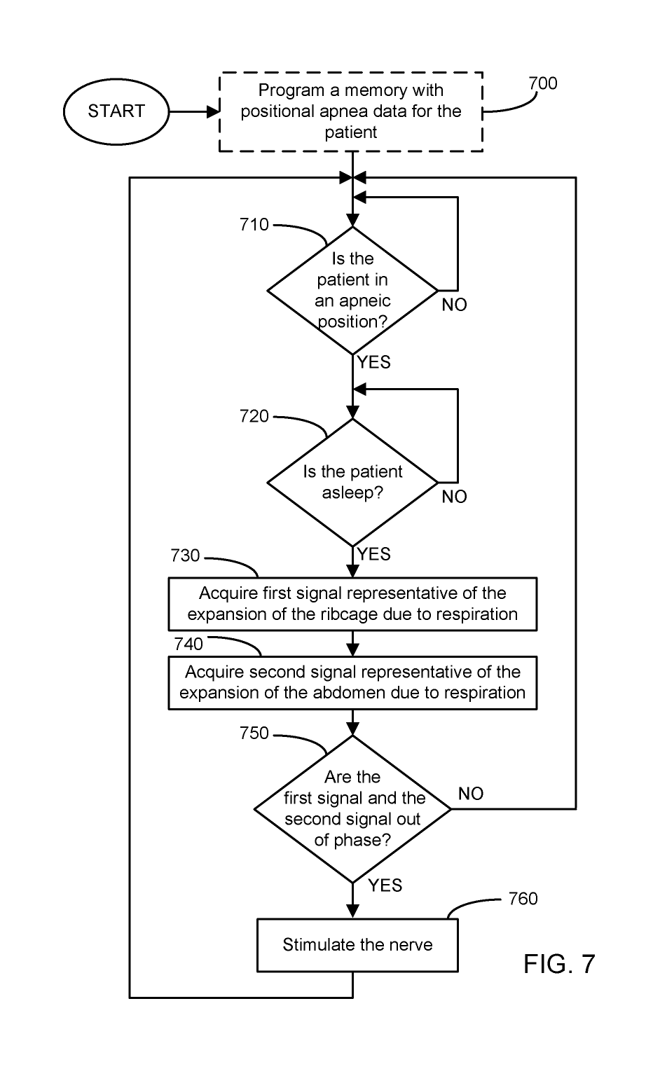

FIG. 7 is a flowchart depicting a method of treating sleep apnea. In embodiments, a system such as one of the systems described above operates according to the following method.

Initially, in some embodiments, a memory in a device is programmed (700) with positional sleep apnea data for the patient.

In some embodiments, it is determined (710) whether the patient is in an apneic position. An apneic position is a position in which the patient is likely to experience apneic events. The most common apneic position is supine, but can include left side, right side, or both. This may be accomplished by monitoring a body position signal from an accelerometer, a gyroscope, a combination of an accelerometer and a gyroscope, or another body position sensor. The method does not progress beyond this step until it is determined that the patient is in an apneic position. Once it is determined that the patient is in an apneic position, the method proceeds to the next step. This embodiment is particularly useful in patients with positional sleep apnea; as these patients experience significantly more apneic events while in particular positions, the subsequent steps can be unnecessary unless in those particular positions. In some embodiments, positional sleep apnea data for the patient is retrieved from a memory, and the positional sleep apnea data and the body position signal are used to determine (710) whether the patient is in an apneic sleeping position. In some embodiments, it is determined (720) whether the patient is asleep. This may be accomplished by monitoring an accelerometer or another sleep sensor. The method does not progress beyond this step until it is determined that the patient is asleep. Once it is determined that the patient is asleep, the method proceeds to the next step.

A first signal representative of the expansion of the ribcage due to respiration is acquired (730). A second signal representative of the expansion of the abdomen due to respiration is acquired (740). Once the first signal and the second signal are acquired, the two signals are compared (750). If the first signal and the second signal are in phase, the method starts over. If the first signal and the second signal are out of phase, a nerve innervating an upper airway muscle is stimulated (760).

In embodiments, an inspiratory portion of respiration is identified. This is the portion of the respiratory cycle during which the patient is attempting to breathe in. Although the patient will not actually be breathing in due to the apneic event, the attempt to breathe in will be present in the second signal, so this portion of the respiratory cycle can still be identified. When the nerve is stimulated (760), the stimulation is applied during the identified inspiratory portion of respiration.

In some alternative embodiments, the step of determining (720) whether the patient is asleep is performed after acquiring at least one of the first signal (730) or the second signal (740). The variance of one or more of the breath-to-breath amplitude or breath-to-breath frequency of one or both signals is monitored. A high variance indicates that the patient is awake and a low variance indicates that the patient is asleep. If, based on the measured variance, it is not determined that the patient is asleep, the method starts over. If it is determined that the patient is asleep, the method proceeds to comparing (750) the two signals.

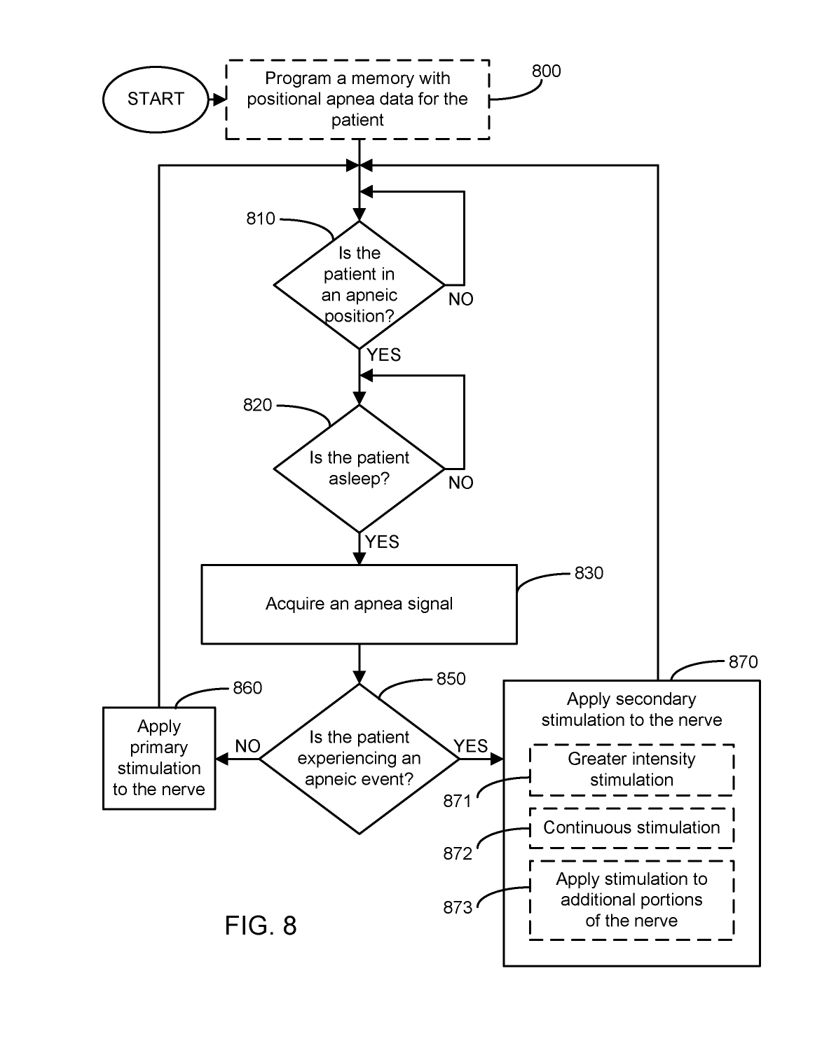

FIG. 8 is a flowchart depicting a method of treating sleep apnea. In embodiments, a system such as one of the systems described above operates according to the following method.

Initially, in some embodiments, a memory in a device is programmed (800) with positional sleep apnea data for the patient.

In some embodiments, it is determined (810) whether the patient is in an apneic position. An apneic position is a position in which the patient is likely to experience apneic events. The most common apneic position is supine, but can include left side, right side, or both. The method does not progress beyond this step until it is determined that the patient is in an apneic position. Once it is determined that the patient is in an apneic position, the method proceeds to the next step. This embodiment is particularly useful in patients with positional sleep apnea; as these patients experience significantly more apneic events while in particular positions, the subsequent steps can be unnecessary unless in those particular positions. In some embodiments, it is determined (820) whether the patient is asleep. The method does not progress beyond this step until it is determined that the patient is asleep. Once it is determined that the patient is asleep, the method proceeds to the next step.

An apnea signal is acquired (830). An apnea signal can be any signal representative of whether the patient is having an apneic event. In embodiments, the apnea signal can be the phase difference between ribcage expansion and abdomen expansion, as discussed above. Alternative embodiments are contemplated, such as a signal from a pressure sensor in the thoracic wall measuring negative pressure resulting from negative pressure in the thoracic cavity. It is determined (850), based on the apnea signal, whether the patient is experiencing an apneic event. If the patient is not experiencing an apneic event, primary stimulation is applied (860) to a nerve innervating an upper airway muscle. If the patient is experiencing an apneic event, secondary stimulation is applied (870) to a nerve innervating an upper airway muscle.

Different embodiments are contemplated for primary stimulation and secondary stimulation. In one embodiment (871), applying secondary stimulation comprises applying greater intensity stimulation than the stimulation used in primary stimulation. The greater intensity may be greater amplitude, greater pulse width, higher frequency, or some combination thereof.

In an embodiment, primary stimulation is stimulation with an intensity which is suitable to provide tonus in the upper airway muscle and secondary stimulation is stimulation with an intensity which is suitable to cause bulk muscle movement in the upper airway muscle. When the first signal and the second signal are in phase, indicating that the airway is not obstructed, stimulation is applied which promotes patency of the airway but does not cause the muscle to actually contract. When the first signal and the second signal are out of phase, indicating that the airway is obstructed, stimulation is applied which causes the upper airway muscle to contract, thereby clearing the airway.

In another embodiment (872), an inspiratory portion of respiration is identified. This is the portion of the respiratory cycle during which the patient is attempting to breathe in. In this embodiment, primary stimulation is stimulation applied during the inspiratory portion of the respiratory cycle (including beginning slightly before the inspiratory period and running throughout the inspiratory period) and secondary stimulation is stimulation applied continuously for a period greater than one inspiratory period. The secondary stimulation may be applied for a period longer than the duration of one full breath. The secondary stimulation may be applied for a set amount of time, such as 30 seconds, before the method returns to the other steps.

In another embodiment (873), primary stimulation is stimulation applied to a first set of fascicles of the nerve and secondary stimulation is stimulation applied to a second set of fascicles of the nerve. Basically, the secondary stimulation is stimulation applied to additional or different portions of the same nerve to thereby affect additional or different portions of the upper airway. This can be accomplished using a multi electrode nerve cuff and current steering.

In an embodiment, acquiring (830) the apnea signal is acquiring the phase difference between ribcage expansion and abdomen expansion, as discussed above, and applying secondary stimulation (870) includes varying the intensity of the stimulation applied based on the phase difference. In particular, the step may include applying higher intensity stimulation for higher phase difference.

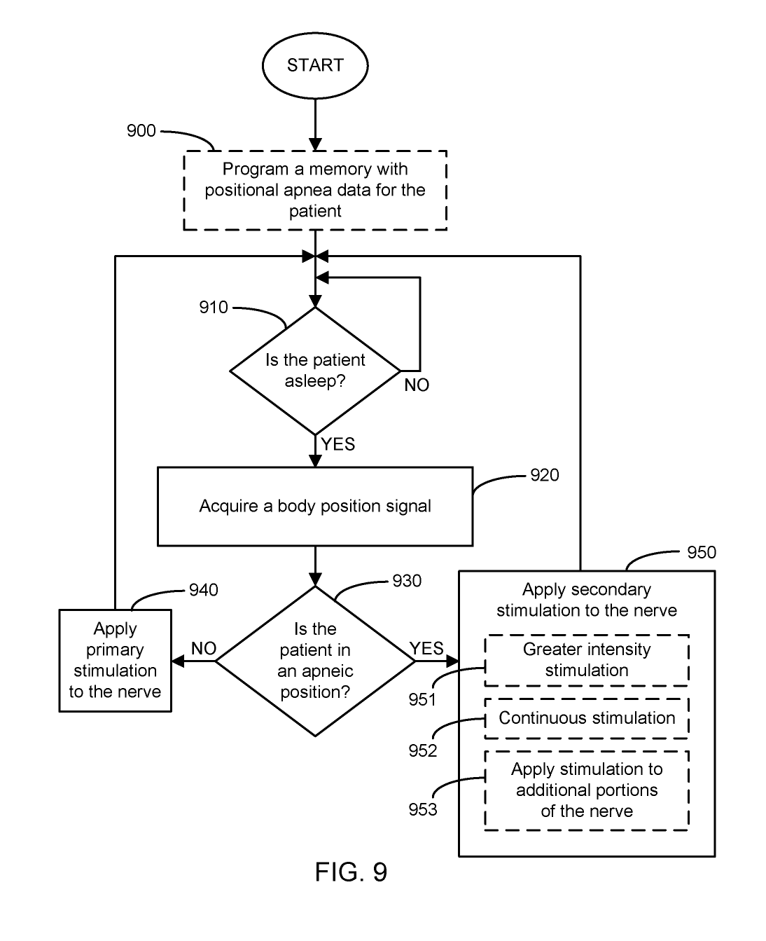

FIG. 9 is a flowchart depicting a method of treating sleep apnea. In embodiments, a system such as one of the systems described above operates according to the following method.

Initially, in some embodiments, a memory in a device is programmed (900) with positional sleep apnea data for the patient.

In some embodiments, it is determined (910) whether the patient is asleep. The method does not progress beyond this step until it is determined that the patient is asleep. Once it is determined that the patient is asleep, the method proceeds to the next step.

A body position signal is acquired (920). The body position signal is representative of whether the patient is lying down in the supine, left side, right side, or prone position. It is determined (930) based on the body position signal whether the patient is in an apneic position. An apneic position is a position in which the patient is likely to experience apneic events. Patients with positional sleep apnea experience significantly more apneic events when supine than when on their left side, on their right side, or prone. They may additionally experience more apneic events while on their left side than right side, or vice versa. Which positions are apneic positions may be configured for a given patient based on sleep study data. If the patient is not in an apneic position, primary stimulation is applied (940) to a nerve innervating an upper airway muscle. If the patient is in an apneic position, secondary stimulation is applied (950) to a nerve innervating an upper airway muscle.

Different embodiments are contemplated for primary stimulation and secondary stimulation. In one embodiment (951), applying secondary stimulation comprises applying greater intensity stimulation than the stimulation used in primary stimulation. The greater intensity may be greater amplitude, greater pulse width, higher frequency, or some combination thereof.

In an embodiment, primary stimulation is stimulation with an intensity which is suitable to provide tonus in the upper airway muscle and secondary stimulation is stimulation with an intensity which is suitable to cause bulk muscle movement in the upper airway muscle. When the first signal and the second signal are in phase, indicating that the airway is not obstructed, stimulation is applied which promotes patency of the airway but does not cause the muscle to actually contract. When the first signal and the second signal are out of phase, indicating that the airway is obstructed, stimulation is applied which causes the upper airway muscle to contract, thereby clearing the airway.

In another embodiment (952), an inspiratory portion of respiration is identified. This is the portion of the respiratory cycle during which the patient is attempting to breathe in. In this embodiment, primary stimulation is stimulation applied during the inspiratory portion of the respiratory cycle (including beginning slightly before the inspiratory period and running throughout the inspiratory period) and secondary stimulation is stimulation applied continuously for a period greater than one inspiratory period. The secondary stimulation may be applied for a period longer than the duration of one full breath. The secondary stimulation may be applied for a set amount of time, such as 30 seconds, before the method returns to the other steps.

In another embodiment (953), primary stimulation is stimulation applied to a first set of fascicles of the nerve and secondary stimulation is stimulation applied to a second set of fascicles of the nerve. Basically, the secondary stimulation is stimulation applied to additional or different portions of the same nerve to thereby affect additional or different portions of the upper airway. This can be accomplished using a multi electrode nerve cuff and current steering.

The foregoing description, for purpose of explanation, has been described with reference to specific implementations. However, the illustrative discussions above are not intended to be exhaustive or to limit the claims to the precise forms disclosed. Many modifications and variations are possible in view of the above teachings. The implementations were chosen and described in order to best explain principles of operation and practical applications, to thereby enable others skilled in the art.

We claim:

1. A system for treating obstructive sleep apnea comprising:
an apnea sensor configured to generate an apnea signal;
a stimulator configured to deliver stimulation to a nerve which innervates an upper airway muscle; and
a controller coupled to the apnea sensor and the stimulator;
wherein the controller is configured to receive the apnea signal from the apnea sensor;
wherein the controller is configured to determine whether an apneic event is detected based on the apnea signal;
wherein the controller is configured to cause the stimulator to apply primary simulation to the nerve if no apneic event is detected; and
wherein the controller is configured to cause the stimulator to apply secondary stimulation to the nerve upon detecting an apneic event.

2. The system of claim 1 wherein primary stimulation and secondary stimulation are both stimulation applied during the inspiratory portion of respiration.

3. The system of claim 1 wherein primary stimulation is stimulation applied during the inspiratory portion of respiration and wherein secondary stimulation is stimulation applied continuously for a period of time greater than the duration of one full breath.

4. The system of claim 1 wherein secondary stimulation is selected from the group consisting of:
stimulation with greater amplitude than primary stimulation;
stimulation with greater pulse width than primary stimulation;
stimulation with higher frequency than primary stimulation; and
stimulation with a combination of two or more of greater amplitude than primary stimulation, greater pulse width than primary stimulation, and higher frequency than primary stimulation.

5. The system of claim 1 wherein primary stimulation is stimulation applied to a first set of fascicles of the nerve and secondary stimulation is stimulation applied to a second set of fascicles of the nerve.

6. The system of claim 1 wherein primary stimulation is stimulation which promotes muscle tone in the upper airway muscle and secondary stimulation is stimulation which causes bulk muscle movement in the upper airway muscle.

7. The system of claim 1 wherein the apnea sensor is selected from the group consisting of:
a first sensor configured to generate a first signal corresponding to movement of the ribcage of the patient during respiration and a second sensor configured to generate a second signal corresponding to movement of the abdomen of the patient during respiration, wherein the apnea signal is the phase difference between the first signal and the second signal; and
a pressure sensor configured to generate a signal representative of the negative pressure in the thoracic cavity.

8. The system of claim 1 wherein
the apnea signal is the phase difference between a first signal corresponding to movement of the ribcage of the patient during respiration and a second signal corresponding to movement of the abdomen of the patient during respiration;
the stimulator is configured to apply secondary stimulation to the nerve at a variable intensity; and
the controller is configured to set the variable intensity based on the phase difference between the first signal and the second signal.

9. A method of treating obstructive sleep apnea comprising:
acquiring an apnea signal;
determining whether an apneic event is detected based on the apnea signal;
applying primary stimulation to a nerve innervating an upper airway muscle when an apneic event is not detected; and
applying secondary stimulation to the nerve innervating an upper airway muscle upon detecting an apneic event.

10. The method of claim 9 wherein primary stimulation is stimulation applied during the inspiratory portion of respiration and wherein secondary stimulation is stimulation applied continuously for a period of time greater than the duration of one full breath.

11. The method of claim 9 wherein secondary stimulation is selected from the group consisting of:
stimulation with greater amplitude than primary stimulation;
stimulation with greater pulse width than primary stimulation;
stimulation with higher frequency than primary stimulation; and
stimulation with a combination of two or more of greater amplitude than primary stimulation, greater pulse width than primary stimulation, and higher frequency than primary stimulation.

12. The method of claim 9 wherein primary stimulation is stimulation applied to a first set of fascicles of the nerve and secondary stimulation is stimulation applied to a second set of fascicles of the nerve.

13. The method of claim 9 wherein primary stimulation is stimulation which promotes muscle tone in the upper airway muscle and secondary stimulation is stimulation which causes bulk muscle movement in the upper airway muscle.

14. The method of claim 9 wherein the apnea signal is acquired from a sensor selected from the group consisting of:
a first sensor configured to generate a first signal corresponding to movement of the ribcage of the patient during respiration and a second sensor configured to generate a second signal corresponding to movement of the abdomen of the patient during respiration, wherein the apnea signal is the phase difference between the first signal and the second signal; and
a pressure sensor configured to generate a signal representative of the negative pressure in the thoracic cavity

15. The method of claim 9 wherein:
the apnea signal is the phase difference between a first signal corresponding to movement of the ribcage of the patient during respiration and a second signal corresponding to movement of the abdomen of the patient during respiration;
secondary stimulation is applied to the nerve at a variable intensity; and
the variable intensity is set based on the phase difference between the first signal and the second signal.
